(19) United States
(12) Patent Application Publication
(10) Pub. No.: US 2024/0091400 A1
(43) Pub. Date: Mar. 21, 2024

(54) ANTIMICROBIAL DRESSING FOR WOUNDS

(71) Applicants: Chander Shekhar, New Delhi (IN); Khalid Khan, New Delhi (IN); Shruti Kaushik, New Delhi (IN)

(72) Inventors: Chander SHEKHAR, NEW DELHI (IN); Farhaan Jalees AHMAD, NEW DELHI (IN)

(21) Appl. No.: 18/518,750

(22) Filed: Nov. 24, 2023

Related U.S. Application Data
(63) Continuation-in-part of application No. 17/266,667, filed on Feb. 8, 2021, now Pat. No. 11,872,240.

(30) Foreign Application Priority Data
Aug. 6, 2018 (IN) 201811029497

(51) Int. Cl. A61L 15/62; A61K 31/7036; A61K 38/14; A61K 47/10; A61K 47/22; A61K 47/28; A61L 15/22; C08L 33/08; C08L 67/04; C08L 77/02

(52) U.S. Cl. CPC A61L 15/62; A61K 31/7036; A61K 38/14; A61K 47/10; A61K 47/22; A61K 47/28; A61L 15/225; C08L 33/08; C08L 67/04; C08L 77/02; A61L 2300/23; A61L 2300/252

(57) ABSTRACT

The present invention relates to an antimicrobial dressing for wounds comprising Vitamin D or Vitamin E as carrier loaded with an antimicrobial agent. More specifically, the present invention relates to antimicrobial wound dressings comprising vitamin D or vitamin E as carrier loaded with an antimicrobial agent for controlling infections and managing potentially infected wounds or infected wounds or treating infected implants used in orthopaedic surgery and various other conditions leading to healing of the wounds and avoidance of development of antibacterial resistance and avoiding complications related to the use of prolonged antibiotics by systemic route.

### CROSS-REFERENCE TO RELATED APPLICATIONS

This application is a continuation in part application of U.S. patent application Ser. No. 17/266,667, filed on Feb. 8, 2021 that claims benefit to Indian Priority Application No. 201811029497 filed on Aug. 6, 2018, which is incorporated by reference herein in its entirety.

### FIELD OF INVENTION

The present invention relates to antimicrobial formulations comprising Vitamin D as carrier loaded with an antibiotic. More specifically, the present invention relates to antimicrobial compositions comprising vitamin D as carrier loaded with an antibiotic for controlling infections and managing potentially infected wounds or infected wounds or treating infected implants used in orthopaedic surgery and various other conditions leading to healing of the wounds and avoidance of development of antibacterial resistance and avoiding complications related to the use of prolonged antibiotics by systemic route.

### BACKGROUND OF THE INVENTION

All types of open infected wounds of the musculoskeletal system are the most prevalent types of infections in injuries and other situations are the leading causes of significant morbidity in all age groups. The standard prophylactic treatment commonly used is systemic antibiotic therapy. However, systemic distribution is reduced in some complex musculoskeletal wounds due to compromised vasculature thus allowing for proliferation of contaminating bacteria leading to infection, thus severely impairs wound healing and can get complicated even when antibiotic resistant and/or biofilm-forming bacterial strains are present resulting in the need for higher concentration of systemic antibiotics causing additional pain and disability to the patient as well as very great economic cost to society in the form of direct medical cost, lost earnings, and disability payments. Further, merely increasing the dosage of systemic antibiotics can help clear infection, but may lead to adverse side effects. The most commonly strains of bacteria responsible for causing wound infections mainly include are S. aureus, S. epidermidis, coagulase-negative staphylococci, and Pseudomonas aeruginosa. It has further been reported that the patients still develop infections even with systemic antibiotics delivered in a clinical setting. For instance, antibiotics present at levels below the minimum inhibitory concentration (MIC) in a S. aureus infection can lead to the development of more resistance to the antibiotic. One could rationally associate the peaks and troughs of antibiotic bioavailability seen in systemic delivery with the development of antibiotic resistant bacterial strains and bio-film.

To further overcome the disadvantages of the systemic antibiotic therapy, a local antibiotic delivery system could increase antibiotic levels at the musculoskeletal wound without increasing risk to the patient.

The most commonly used antibiotics for local delivery to musculoskeletal trauma includes vancomycin and tobramycin. Both antibiotics are considered reliable because they are capable of sustained activity over an extended elution time, storage time, or variable environmental conditions such as a low pH. Vancomycin is effective against severe infections caused due to susceptible methicillin-resistant (beta-lactam resistant) staphylococci in penicillin-allergic patients; patients who cannot receive or have failed to respond to other drugs, including penicillins and cephalosporins; and for infections due to vancomycin- susceptible organisms resistant to other antimicrobials while tobramycin or gentamycin or amikacin has a broad spectrum of efficacy including against Gram negative bacteria, such as the bio-film forming P. aeruginosa.

Further, the commonly used carrier material in local antibiotic delivery systems includes polymethylmethacrylate, bone cement, POP pellets, collagen sponge, fibrin sealant, Hydrosy apatite blocks, Poly-glycolide implants, di Lactate polymer and calcium sulfate, which increases the local antibiotic levels within the tissue surrounding a wound; which could enhance treatment outcome for contaminated wounds. However, the current options present limitations such as surgical removal after a period of time, rapid degradation, or a limited choice of antibiotics utilized at the time of application. Further polymethylmethacrylate (PMMA) provides number of significant disadvantages such as it is dense, acrylic, and non-resorbing material, which generally must be removed in a second surgical procedure when its function has been fulfilled to avoid becoming a nidus for future infection. Also, it can release sub-inhibitory concentrations of drugs over an extended implantation period, and bacteria adherent to PMMA cement thus can acquire resistance to the loaded antibiotic because of long exposure to the sub-inhibitory concentrations. Also, PMMA is not suitable for delivery of thermo-sensitive antibiotics because of the high temperatures generated during PMMA curing.

Development of anti-biotic resistance with prolonged use of antibiotic is well known and the emergence of resistant bacteria is difficult to control and the bio-films which form around the infected sites in infected implants are difficult to eradicate. Therefore, the present limitations provide the need for the development of a novel and cost effective local drug delivery system with suitable bio-carrier in order to provide enhanced treatment over current approaches, particularly in complex trauma wounds or patients with infection risk factors i.e., diabetes, positive skin cultures, history of infection.

### OBJECT OF THE INVENTION

The main object of the present invention is to provide an antimicrobial composition comprising Vitamin D as carrier for local delivery of antibiotics for controlling infections and managing potentially infected wounds or infected wounds or treating infected implants used in orthopedic surgery and various other such indications.

Another object of the present invention is to provide a method of preparing an antimicrobial formulation comprising Vitamin D for local delivery of the drugs preferably, antibiotics is not limited to aminoglycosides, Beta-lactam agents, and Quinolones.

Yet another object of the present invention is to provide an antimicrobial composition comprising Vitamin D as carrier which is very safe, cost-effective, and extremely effective without any complications in controlling and completely eradicating infections.

Still another object of the present invention is to provide an antimicrobial dressing for wound healing that is biodegradable in nature and controls and manages infected wounds.

### SUMMARY OF THE INVENTION

The present invention relates to antimicrobial formulations comprising vitamin D as carrier of local delivery of the drugs preferably, antibiotics. More specifically, the present invention relates to vitamin D as carrier beads for local delivery of the antibiotic drugs/components for controlling infections and managing potentially infected wounds or infected wounds or treating infected implants used in orthopedic surgery and various other such indications.

In an embodiment the present invention provides a pharmaceutical composition comprising of an antimicrobial agent or a pharmaceutically acceptable salt or a combination of one or more antimicrobial agents or a pharmaceutically acceptable salt; a pharmaceutically acceptable carrier; wherein, the antimicrobial agent includes but is not limited to vancomycin, tobramycin; and the pharmaceutically acceptable carrier includes but is not limited to Vitamin D₃.

In another embodiment the present invention provides a wound dressing comprising of at least one antimicrobial agent; at least one pharmaceutically acceptable carrier; at least one pharmaceutically acceptable excipient; and a base; wherein, said antimicrobial agent is tobramycin or vancomycin or a combination thereof; said pharmaceutically acceptable carrier is a fat soluble vitamin; said base is a gel base or a knitted fabric; and said pharmaceutical composition is applied topically for managing an infected open wound within a time span of 1-15 days.

In yet another embodiment the present invention relates to a method of preparing antimicrobial formulation comprising Vitamin D as carrier for local delivery of the drugs, preferably antibiotic comprising steps of: (a) mixing Vitamin D with antibiotic to obtain a mixture in a desired ratio; (b) blending the mixture obtained in step (a) thoroughly to obtain a homogenous mixture; (c) mixing the mixture obtained in step (b) with sterile water to obtain a smooth paste; (d) filling and pressing the smooth paste obtained in step (c) into the cavities of the flexible mold to obtain beads; (e) leaving the beads obtained in step (d) undisturbed and letting it dehydrate until hard; and (f) removing the beads from the flexible mold to obtain Vitamin D carrier beads.

In yet another embodiment the present invention, relates to using Vitamin D as carrier for local delivery of the drugs, preferably antibiotic which is very safe, cost-effective, and extremely effective without any complications in controlling and completely eradicating infections.

In yet another embodiment the present invention, relates to using Vitamin D as carrier for local delivery of the drugs, preferably antibiotics, which is not limited to aminoglycosides, Beta-lactam agents, Quinolones.

In yet another embodiment the present invention, relates to using Vitamin D as carrier of local delivery of the drugs, preferably antibiotics for controlling infections and managing potentially infected wounds or infected wounds or treating infected implants used in orthopedic surgery and various other conditions.

### DETAILED DESCRIPTION OF THE INVENTION

The present invention will now be described more fully hereinafter. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention relates to an antimicrobial formulation comprising Vitamin D as preferred carrier for local delivery of the drugs preferably, antibiotics. More specifically, the present invention relates to vitamin D as carrier for local delivery of the drugs, preferably antibiotics for controlling infections managing potentially infected wounds or infected wounds or treating infected implants used in orthopedic surgery and various other conditions.

In an embodiment, the present invention provides a wound dressing comprising of at least one antimicrobial agent; at least one pharmaceutically acceptable carrier; at least one pharmaceutically acceptable excipient; and a base; wherein, said antimicrobial agent is tobramycin or vancomycin or a combination thereof; said pharmaceutically acceptable carrier is a fat soluble vitamin; said base is a gel base or a knitted fabric; and said pharmaceutical composition is applied topically for managing an infected open wound within a time span of 1-15 days.

The fat soluble vitamin in the wound dressing is Vitamin D or Vitamin E or a combination thereof and the at least one antimicrobial agent is in concentration of 0.1-10 (% w/w). Further, the concentration of vitamin D is in a range of 2000-60000 IU and the concentration of vitamin E is 0.5 (% w/w).

In another embodiment, the present invention provides a pharmaceutical composition comprising of an antimicrobial agent or a pharmaceutically acceptable salt or a combination of one or more antimicrobial agents or a pharmaceutically acceptable salt; a pharmaceutically acceptable carrier; wherein, the antimicrobial agent includes but is not limited to vancomycin, tobramycin; and the pharmaceutically acceptable carrier includes but is not limited to Vitamin D₃. The composition optionally includes but is not limited to buffering agents, preservatives, coloring agents, and stabilizers.

In yet another embodiment, the present invention relates to a pharmaceutically acceptable carrier which includes, but is not limited to, Vitamin D₃ and Vitamin E. Vitamin D₃ is actually a fat-soluble prohormone steroid that has endocrine, paracrine, and autocrine functions. The endocrine effects of vitamin D₃ are mainly involved in serum calcium homeostasis. The paracrine and autocrine effects of vitamin D₃ depend on genetic transcription, unique to the type of cell expressing nuclear vitamin D receptors. Vitamin D₃ loosely binds to the antibiotic which includes but is not limited to tobramycin and get transported to the deeper parts of the skin and connective tissues where it gets released for antimicrobial activity; immunomodulator activity of vitamin is additive to produce fast healing effects in complicated wounds. It also has an important role in modulating the innate and adaptive immune system, which influences the production of important endogenous antimicrobial peptides such as cathelicidin and regulating the inflammatory cascade. Cathelicidin is an antimicrobial peptide that is able to directly kill pathogens or bind to the endotoxins. It is active against viruses and other bacteria like Mycobacterium tuberculosis. Vitamin D₃ also regulates the adaptive immune system particularly T-cells (TH1 cells, TH2 cells, TH17 cells, and T regulatory cells). Finally, vitamin D₃ also regulates the inflammatory cascade by modulating the nuclear factor κB pathway.

In yet another preferred embodiment, the present invention relates to a method of preparing an antimicrobial formulation comprising Vitamin D as carrier for local delivery of the drugs, preferably antibiotics comprising steps of: (a) mixing Vitamin D (calcirol) with antibiotic powder to obtain a mixture in a desired ratio; (b) blending the mixture obtained in step (a) thoroughly to obtain a homogenous mixture; (c) mixing the mixture obtained in step (b) with sterile water to obtain a smooth paste; (d) filling and pressing the smooth paste obtained in step (c) into the cavities of the flexible mold to obtain beads; (e) leaving the beads obtained in step (d) undisturbed and letting it dehydrate until hard; and (f) removing the beads from the flexible mold to obtain Vitamin D carrier beads.

In yet another preferred embodiment, the present invention relates to using Vitamin D as carrier for local delivery of the drugs preferably, antibiotics which is stable, water soluble, active against the most common bacterial pathogens involved in bone and soft tissue infections, is released locally at concentration exceeding several times, usually ten times, have low rate of allergic reaction, low rate of primary resistance, not produce supra infections, is very safe, cost-effective, and extremely effective without any complications in controlling and completely eradicating infections.

In yet another preferred embodiment, the present invention relates to using Vitamin D as carrier for local delivery of the drugs preferably, antibiotics for controlling infections managing potentially infected wounds or infected wounds or treating infected implants used in orthopedic surgery and various other conditions.

In yet another preferred embodiment, the present invention relates to providing Vitamin D as carrier for local delivery of the drugs preferably, antibiotics not limited to Amino glycosides Beta lactam agents, Quinolones and the amino glycosides not limited to tobramycin, gentamycin or vancomycin. Table 1 compares Vitamin D3 Granules versus Currently Used Delivery Carriers in Local Antibiotic Delivery System. Though several biodegradable delivery systems have been developed, such as calcium sulfate, collagen sponges, calcium phosphate, and polylactic acid, each of these methods have specific disadvantages as shown in Table 1. Degradable collagen fleeces and calcium-based carriers are known to release large amount of antibiotics in the first 24 hours after implantation. While prolonged release is not guaranteed, they absorb large amount of water, stimulate seromas, and increase the risk of secondary infection. Polyglycolic acid carriers are known to produce acidic degradation products, which can lead to resorption of the bone. Considering the bacteriological finding in bone and soft tissue infections that is chronic osteomyelitis caused by Staphylococcus aureus, Enterobacteriaceae, and Pseudomonas aeruginosa, the most acceptable agent in local delivery systems are aminoglycosides and to a lesser extent various β-lactam agents and quinolones.

TABLE 1
Vitamin D₃ Granules versus Currently Used Delivery Carriers in Local Antibiotic
Delivery System

| | | | Promote | | Increase | |
|---|---|---|---|---|---|---|
| | | | Tissue | Fat | Bone Mineral | |
| Carrier | Antibiotic | Biodegradable | Growth | Soluble | Density | Side Effects |
| PMMA beads¹² | Gentamycin, tobramycin, vancomycin | x | x | x | x | Secondary infection |
| Calcium sulfate | Tobramycin | ✓ | x | x | — | Emergence of |
| | | | | | | resistant bacteria |
| Collagen sponges | Gentamycin, tobramycin, vancomycin | ✓ | x | x | x | Can contain impurities |
| Polylactic acid | Gentamycin, tobramycin, vancomycin | ✓ | x | x | x | Local tissue reaction |
| Vitamin D₃ | Gentamycin, tobramycin, vancomycin | ✓ | ✓ | ✓ | ✓ | None |
| granules | | | | | | |

In yet another preferred embodiment of the present invention, the antimicrobial composition comprising 1 gm Vitamin D₃ carrier and 5-20 mg of an antibiotic drug, or alternately can have higher possible carrier to drug ratio in range of 100:1 to 1:1, or depending on the drug composition, the ratio can vary to modulate bioavailability and least cytotoxicity.

In yet another preferred embodiment of the present invention, the antimicrobial formulation is formulated for delayed/controlled drug delivery, wherein, the delivery is delayed in the range of 1 to 15 days.

In yet another preferred embodiment, the present invention relates to using Vitamin D as carrier for local delivery of drugs preferably, antibiotics through various modes of drug carrier delivery systems not limited to Polymer based nanoparticles, liposomes or phospholipid vesicles and other colloidal systems which involves micro or nano encapsulation of Vitamin D along with the drug. Depending on the hydrophobic nature of Vitamin D, a hydrophobic polymer matrix is crucial to the drug loading and the most PLGA is identified as primary choice which has competence to encapsulate drugs of poor solubility.

In yet another preferred embodiment, the present invention provides a wound dressing in which the antimicrobial agent loaded nanoemulsion to a gel base. The gel base is carbopol 934.

In yet another preferred embodiment, the present invention provides a wound dressing wherein the base is a knitted fabric that is a perforated material formed of a nylon multifilament.

In yet another embodiment, the present invention provides a wound dressing that is a biodegradable wound dressing, which further comprises of a polymeric scaffold loaded with the antimicrobial agent and pharmaceutically acceptable carrier. The polymeric scaffold is of poly(lactic acid) and the polymeric scaffold and antimicrobial agent forms a conjugate in a range of 10-20% w/v of the wound dressing.

In yet another preferred embodiment, the present invention relates to antimicrobial formulation comprising Vitamin D as carrier wherein the mode of administration of the composition is not limited to enteral route, parenteral route, topical route or arterial route. The enteral route mainly comprises of oral, sublingual and rectal while the parenteral route comprises of intravenous, intramuscular, subcutaneous and inhalation. Further, the topical route comprises skin, mucous membrane, mouth and pharynx, eyes, ears, nose and gastrointestinal tract while in the arterial route the drug is injected into the artery that is supplying the blood to the desired site so that localized action is achieved.

In yet another preferred embodiment, the present invention provides an ointment composition having Formula I comprising the ingredients including 2000-60,000 IU of Vitamin D, 0.1-10% w/w of Vancomycin, 0.1-10% w/w of Tobramycin, 0.01-30.00% w/w of N-Methyl Pyrolidone (Pharmasolve), 20.0-99.90% w/w of White wax (White bees wax), 14.40% w/w of Mineral oil (Liquid Paraffin), 3.00% w/w of Paraffin (Hard Paraffin) and 70.80% w/w of Petrolatum, white. Further, the composition is prepared by the followings steps of:
a) Dissolving Vitamin D in mineral oil to obtain a tacrolimus solution;
b) Melting white wax, mineral oil, paraffin and white petrolatum together at 70° C. to obtain a molten mass;
c) Cooling the molten mass obtained in step (b) to 40° C.;
d) Adding the tacrolimus solution of step (a) to the molten mass of step (c) under gentle stirring to obtain a mixture of mass; and
e) Stirring the mixture of mass of step (d) gently for 5 minutes to obtain a pharmaceutical ointment composition having optimum and uniform consistency.

In yet another preferred embodiment, the present invention provides an ointment composition having formula II comprising the ingredients including 2000-60,000 IU of Vitamin D, 0.1-10% w/w of Vancomycin, 0.1-10% w/w of Tobramycin, 0.01-30.00% w/w of N-Methyl Pyrolidone (Pharmasolve), 20.0-99.90% w/w of white wax (white bees wax), 14.40% w/w of mineral oil (liquid paraffin), 3.00% w/w of paraffin (hard paraffin) and 74.30% w/w of petrolatum, white. Further, this composition was prepared in substantially the same manner as the composition of Formula I, except that (1) white wax was used to increase the consistency of the ointment base and (2) hard paraffin was used as the stiffening agent in the ointment base.

In yet another preferred embodiment, the present invention provides a topical, medicinal spray composition comprising a drug or combination of drugs in a carrier which, when sprayed on a surface, forms a film. The composition comprises at least one medicament, at least one film former and at least one vehicle. The composition of the invention further comprise at least one permeation enhancer, at least one solubilizer, at least one plasticizer, and at least one water-soluble additive. A metered dose of the composition is sprayed on a topical site to form a stable, breathable film, preferably over a fixed surface area. A wide range of medicaments for human and veterinary use are present that act locally or transdermally.

Further, the ingredients of topical composition 1 include 2000 IU of Vitamin D, 0.3% w/w of Vancomycin, 0.3% w/w of Tobramycin, 2.25% w/w of Plastoid B, 0.25% w/w of Eudragit E 100, 3.0% w/w of Propylene glycol, 3.5% w/w of sodium lauryl sulfate, 20% w/w of acetone, propellant, 0.1% w/w of Vitamin E and 1.0% w/w of Transcutol.

Furthermore, the ingredients of topical composition 2 include 2000 IU of Vitamin D, 0.3% w/w of Vancomycin, 0.3% w/w of Tobramycin, 3% w/w of Povidone, 2% w/w of Povidone VA-64, 0.5% w/w of Vitamin E, 1.0% w/w of PEG 400, 1.5% w/w of Propylene glycol, 15% w/w of Ethanol, 15% w/w of acetone and propellant.

Moreover, the ingredients of topical composition 3 include 2000 IU of Vitamin D, 0.3% w/w of Vancomycin, 0.3% w/w of Tobramycin, 6% w/w of PVP-K-30, 4% w/w of PVP VA, 1% w/w of Vitamin E, 2% w/w pf PEG 6000, 3% w/w of Propylene glycol, 58.1% w/w of P12 and 24.9% w/w of P11.

In yet another embodiment, the present invention provides a wound dressing by Carbopol 934 gel base having ingredients including 2000 IU of Vitamin D, 0.3% w/w of Vancomycin, 0.3% w/w of Tobramycin, 1% w/w of Carbopol 934, 0.5% w/w of Vitamin E, 0.5% w/w of PEG 400, 1.0% w/w of Propylene glycol, 1.5% w/w of Transcutol. Further, a wound dressing was produced in accordance with the production method described above. Specifically, the wound dressing was produced as follows. Drug loaded nanoemulsion was added into the gel base. The prepared gel was prepared using Carbopol 934 as gel base.

In yet another embodiment, the present invention provides the ingredients including 2000 IU of Vitamin D, 0.3% w/w of Vancomycin, 0.3% w/w of Tobramycin, 1% w/w pf Carbopol 934, 0.5% w/w of PEG 300, 1.0% w/w of Propylene glycol. Further, a wound dressing was obtained by a perforated material in which the average opening area of through-holes and the average through-hole number were different from those of the present invention. The knitted fabric constituting the perforated material used was formed of a nylon multifilament.

In yet another preferred embodiment, the present invention relates to antimicrobial formulation comprising Vitamin D granules as carrier and excipients. Pharmaceutical drug delivery systems consist of additional therapeutically inactive constituents other than APIs which are required for the formulation to work in the biological system, are called as excipients. Excipients have a broad range of functionalities in making formulations in the form of bulking agents, binders, disintegrants, flavors, glidants, lubricants, preservatives, permeation enhancers, solubility enhancers, preservatives and sweeteners, etc. Further to enhance the drug bioavailability, excipients are used which enhance the stability and increases the solubility of the drug. Solubility enhancing excipients are mainly categorized into three sections called as a polymer, surfactant, and lipid based. However, polymer based excipients are widely used for solubility enrichment process. The surfactants in surfactant based excipient can facilitate their solubilization. Further, surfactants can solubilize the poorly soluble drug molecules by micelle formation or by acting as co-solvents. The combination of lipophilic surfactants, hydrophilic surfactants, water-soluble co-solvents, triglyceride oils, co-surfactants can build the efficient and stable self-emulsifying drug delivery system which can improve drug solubility and oral absorption.

In yet another preferred embodiment, the Vitamin D carrier for local delivery of drugs preferably, antibiotics is formulated in any dosage form suitable for topical administration preferably, the composition is in a form such as a cream, ointment, gel, lotion, foam, powder, aerosol, spray, or liquid solution or may be in form of a polymeric patch containing drug preferably, anti-biotic which facilitates transdermal delivery for treating the infection which aids in managing the release of drug for longer period of time in a sustained manner. The topical administration is given for open wounds not limited to incisions or incised wounds, lacerations, abrasions, avulsions, puncture wounds, penetration wounds and gunshot wounds. Further, the topical administration can also be used in cases of burns.

The local delivery of the antibiotics using Vitamin D as carrier is applicable in compound injuries of the limbs sustained in road traffic accidents, non-healing infected wounds, post-operative patients in orthopedics with infected implants with open discharging wounds which are not healing, in surgical situations where there occurs formation of dead spaces in deep layers or superficial layers of the wounds with possibility of formation of infected hematoma, in degloving wounds, in surgeries where the flaps whether vascularised flaps or rotation flaps or facio-cutaneous flaps are required to cover the defects of the soft tissues and the bone, in situations where plates & screws are being used especially when the injuries are compound in nature. Further, at various sites in general surgical conditions where the patient immunity is low, where the patient is severely diabetic and on several medications like disease-modifying anti-rheumatic drugs (DMRD) in rheumatoid arthritis or immune-suppressive drugs in other conditions and other miscellaneous conditions where the treating surgeon decides according to his personal experiences.

In yet another preferred embodiment of the present invention, the composition comprises effective amount of mixture of at least two antibiotic compounds such as vancomycin and tobramycin loaded on Vitamin D granules. The composition when applied directly onto the affected area, with or without repeating the dosage, shows quick recovery and antibiotic efficacy in shorter period of time.

In yet another preferred embodiment of the present invention, the composition comprises effective amount of mixture of at least two antibiotic compounds such as vancomycin and tobramycin loaded on Vitamin D granules. The ratio of antibiotics to Vitamin D may vary depending upon the type, location and severity of infection in the wounds. The amount of antibiotic dosage with respect to the amount of Vitamin D is increased in case of chronic wounds. However, in case of acute severity of the infection the amount of antibiotic dosage with respect to the amount of Vitamin D is reduced. Therefore, the amounts of antibiotics used in the composition as compared to the amounts of Vitamin D used in wound dressings may vary and is altered according to the severity of infection and type of wound encountered.

In an alternate embodiment, the present invention provides a composition comprising effective amount of vancomycin loaded on Vitamin D granules. The ratio of vancomycin with respect to the Vitamin D used in wound dressings may vary and is altered according to the severity of infection and type of wound encountered.

In another alternate embodiment, the present invention provides a composition comprising effective amount of tobramycin loaded on Vitamin D granules. The ratio of vancomycin with respect to the Vitamin D used in wound dressings may vary and is altered according to the severity of infection and type of wound encountered.

In an exemplary procedure for treating an infectious wound of size 3×3.5 sq. inch, 1 gm Vitamin D₃ granules i.e. equivalent to 60,000 IU were mixed with 60 mg Tobramycin. The mixture was applied to the wound resulting is complete healing within 4-5 days.

In another exemplary procedure for treating an infectious wound with pus, 1 gm Vitamin D₃ granules i.e. equivalent to 60,000 IU were mixed with 60 mg Tobramycin and 10-20 mg Vancomycin. The mixture was applied to the wound resulting is complete healing within 3-4 days.

In another exemplary procedure for treating an infectious wound of substantial size having moderate pus, 1 gm Vitamin D3 granules i.e. equivalent to 60,000 IU were mixed with 60 mg Tobramycin and 5-10 mg Vancomycin. The mixture was applied to the wound resulting is complete healing within 4-5 days.

In another exemplary procedure for treating an infectious wound of substantial size having moderate pus, 1 gm Vitamin D3 granules i.e. equivalent to 60,000 IU were mixed with 5-10 mg Vancomycin. The mixture was applied to the wound resulting is complete healing within 4-5 days.

### EXAMPLE 1

### For Experimentation Data

### Ointment Composition

### Ointment—Formula Composition I

### Materials

The ingredients used in this example are set forth below in Table 2.

TABLE 2
Ingredients of the ointment for Formula Composition I

| | | | Range which |
|---|---|---|---|
| | | % w/w | is used |
| Ingredients | Category | (in Example 1) | (% w/w) |
| Vitamin D | API | 2000 IU | 2000-60,000 IU |
| Vancomycin | API | 0.3 | 0.1-10 |
| Tobramycin | API | 0.3 | 0.1-10 |
| N-Methyl | Solubilizer & | 5.00 | 0.01-30.00 |
| Pyrolidone | Penetration | | |
| (Pharmasolve) | Enhancer | | |
| White wax | Ointment | 6.70 | 20.0-99.90 |
| (White bees wax) | Base/Emollient | | |
| Mineral oil | | 14.40 | |
| (Liquid Paraffin) | | | |
| Paraffin | | 3.00 | |
| (Hard Paraffin) | | | |
| Petrolatum, White | | 70.80 | |
| Total | | 100.00 | |

### Method

The composition of this example was prepared as follows: Dissolving Vitamin D in mineral oil to obtain a tacrolimus solution;
b) Melting white wax, mineral oil, paraffin and white petrolatum together at 70° C. to obtain a molten mass;
c) Cooling the molten mass obtained in step (b) to 40° C.;
d) Adding the tacrolimus solution of step (a) to the molten mass of step (c) under gentle stirring to obtain a mixture of mass; and
e) Stirring the mixture of mass of step (d) gently for 5 minutes to obtain a pharmaceutical ointment composition having optimum and uniform consistency.

### Ointment—Formula Composition II

### Materials

The ingredients used in this example are set forth below in Table 3.

TABLE 3
Ingredients of the ointment for Formula Composition II

| | | | Range which |
|---|---|---|---|
| | | % w/w | is used |
| Ingredients | Category | (in Example 2) | (% w/w) |
| Vitamin D | API | 2000 IU | 2000-60,000 IU |
| Vancomycin | API | 0.3 | 0.1-10 |
| Tobramycin | API | 0.3 | 0.1-10 |
| N-Methyl | Solubilizer & | 1.50 | 0.01-30.00 |
| Pyrolidone | Penetration | | |
| (Pharmasolve) | Enhancer | | |
| White wax | Ointment Base | 6.70 | 20.0-99.90 |
| (White bees wax) | | | |
| Mineral oil | | 14.40 | |
| (Liquid Paraffin) | | | |
| Paraffin | | 3.00 | |
| (Hard Paraffin) | | | |
| Petrolatum, White | | 74.30 | |
| Total | | 100.00 | |

### Method

The composition of this example was prepared in substantially the same manner as the composition of Formula Composition I, except that (1) white wax was used to increase the consistency of the ointment base and (2) hard paraffin was used as the stiffening agent in the ointment base.

### EXAMPLE 2

### Topical Spray Composition

A topical, medicinal spray composition is provided comprising a drug or combination of drugs in a carrier which, when sprayed on a surface, forms a film. The composition comprises at least one medicament, at least one film former and at least one vehicle. The composition of the invention further comprise at least one permeation enhancer, at least one solubilizer, at least one plasticizer, and at least one water-soluble additive. A metered dose of the composition is sprayed on a topical site to form a stable, breathable film, preferably over a fixed surface area. A wide range of medicaments for human and veterinary use are present that act locally or transdermally. The composition is provided in the below tables.

### Composition 1:

| Ingredients | Percent w/w |
|---|---|
| Vitamin D | 2000 IU |
| Vancomycin | 0.3 |
| Tobramycin | 0.3 |
| Plastoid B | 2.25 |
| Eudragit E 100 | 0.25 |
| Propylene glycol | 3.0 |
| Sodium lauryl sulfate | 3.5 |
| Acetone | 20 |
| Propellant | q.s. |
| Vitamin E | 0.1 |
| Transcutol | 1.0 |

### Composition 2:

| Ingredients | Percent w/w |
|---|---|
| Vitamin D | 2000 IU |
| Vancomycin | 0.3 |
| Tobramycin | 0.3 |
| Povidone | 3 |
| Povidone VA-64 | 2 |
| Vitamin E | 0.5 |
| PEG 400 | 1.0 |
| Propylene glycol | 1.5 |
| Ethanol | 15 |
| Acetone | 15 |
| Propellant | q.s. |

### Composition 3:

| Ingredients | Percent w/w |
|---|---|
| Vitamin D | 2000 IU |
| Vancomycin | 0.3 |
| Tobramycin | 0.3 |
| PVP-K-30 | 6 |
| PVP VA | 4 |
| Vitamin E | 1 |
| PEG 6000 | 2 |
| Propylene glycol | 3 |
| P12 | 58.1 |
| P11 | 24.9 |

### EXAMPLE 3

### Wound Dressing Composition

### Wound Dressing by Carbopol 934 gel base

### Composition 1:

| Ingredients | Percent w/w |
|---|---|
| Vitamin D | 2000 IU |
| Vancomycin | 0.3 |
| Tobramycin | 0.3 |
| Carbopol 934 | 1 |
| Vitamin E | 0.5 |
| PEG 400 | 0.5 |
| Propylene glycol | 1.0 |
| Transcutol | 1.5 |

### Method

A wound dressing was produced in accordance with the method described above. Specifically, the wound dressing was produced by adding the drug loaded nanoemulsion into the gel base. The gel was prepared using Carbopol 934 as gel base.

### Wound Dressing by a perforated material

### Composition 2:

| Ingredients | Percent w/w |
|---|---|
| Vitamin D | 2000 IU |
| Vancomycin | 0.3 |
| Tobramycin | 0.3 |
| Carbopol 934 | 1 |
| PEG 300 | 0.5 |
| Propylene glycol | 1.0 |

### Method

A wound dressing was obtained by a perforated material in which the average opening area of through-holes and the average through-hole number were different from those of the present invention. The knitted fabric constituting the perforated material used was formed of a nylon multifilament.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A wound dressing comprising of:
at least one antimicrobial agent;
at least one pharmaceutically acceptable carrier;
at least one pharmaceutically acceptable excipient; and
a base;
wherein,
said antimicrobial agent is tobramycin or vancomycin or a combination thereof;
said pharmaceutically acceptable carrier is a fat soluble vitamin;
said base is a gel base or a knitted fabric; and
said pharmaceutical composition is applied topically for managing an infected open wound within a time span of 1-15 days.

2. The wound dressing according to claim 1, wherein said fat soluble vitamin is Vitamin D or Vitamin E or a combination thereof.

3. The wound dressing according to claim 1, wherein the at least one antimicrobial agent is in concentration in a range of 0.1-10 (% w/w).

4. The wound dressing according to claim 1, wherein vitamin D is in concentration in a range of 2000-60000 IU.

5. The wound dressing according to claim 1, wherein vitamin D is in concentration in a range of 2000-60000 IU and vitamin E is in a concentration of 0.5 (% w/w).

6. The wound dressing according to claim 1, wherein at least one pharmaceutically acceptable excipient includes buffering agents, preservatives, coloring agents, stabilisers, solubilizers, permeation enhancers, water soluble additives and other pharmaceutical excipients.

7. The wound dressing according to claim 1, wherein said pharmaceutically acceptable excipient is PEG 400 or PEG 300 or propylene glycol or a combination thereof.

8. The wound dressing according to claim 1, wherein the at least one pharmaceutically acceptable excipient is in a concentration range of 1-1.5 (% w/w).

9. The wound dressing according to claim 1, wherein said antimicrobial agent is loaded on nanoemulsion of said pharmaceutically acceptable carrier.

10. The wound dressing according to claim 1, wherein said antimicrobial agent loaded nanoemulsion is added to the gel base.

11. The wound dressing according to claim 1, wherein said gel base is carbopol 934.

12. The wound dressing according to claim 1, wherein said knitted fabric is a perforated material formed of a nylon multifilament.

13. The wound dressing according to claim 1, wherein the antimicrobial agent or a combination thereof and the pharmaceutically acceptable carrier or a combination thereof are in a ratio ranging from 1:100 to 1:1.

14. The wound dressing according to claim 1, wherein said wound dressing is a biodegradable wound dressing, further comprising of a polymeric scaffold loaded with the antimicrobial agent and pharmaceutically acceptable carrier.

15. The wound dressing according to claim 14, wherein said polymeric scaffold is of poly(lactic acid).

16. The wound dressing according to claim 14, wherein said polymeric scaffold and antimicrobial agent forms a conjugate in a range of 10-20% w/v of the wound dressing.
